(19) United States
(12) Patent Application Publication
(10) Pub. No.: US 2024/0058167 A1
(43) Pub. Date: Feb. 22, 2024

(54) AUTOMATED ADMINISTRATION OF THERAPEUTICS TO THE EYE

(71) Applicant: TWENTY TWENTY THERAPEUTICS LLC, South San Francisco, CA (US)

(72) Inventors: Michael Eyal, South San Francisco, CA (US); Timothy Stowe, Alameda, CA (US)

(21) Appl. No.: 18/234,785

(22) Filed: Aug. 16, 2023

Related U.S. Application Data
(60) Provisional application No. 63/398,347, filed on Aug. 16, 2022.
(60) Provisional application No. 63/400,122, filed on Aug. 23, 2022.

(51) Int. Cl. A61F 9/00

(52) U.S. Cl. CPC A61F 9/0008

(57) ABSTRACT

Systems and methods are provided for applying a drop of a therapeutic to an eye of a user. Reflected light from the eye of the user is measured at a sensor to provide a time series of intensity values. It is determined from the time series of intensity values if the eye has blinked. The drop of the therapeutic is released in response to a determination that the eye has blinked.

### RELATED APPLICATIONS

This application claims priority from each of U.S. Provisional Patent Application Ser. No. 63/398,347, filed Aug. 16, 2022, and U.S. Provisional Patent Application Ser. No. 63/400,122, filed Aug. 23, 2022. Each of these applications is hereby incorporated by reference in its entirety.

### TECHNICAL FIELD

The disclosure relates generally to the field of medical systems, and more particularly to automated administration of therapeutics to the eye.

### BACKGROUND

Patients, especially elderly patients, have difficulty applying prescribed medication to their eyes. Most people have an instinctive reaction to blink in response to an approaching eye drop, which blocks the drop from being fully absorbed into the cornea. The resulting incomplete application of the medication to the eye can lead to worsened treatment outcomes.

### SUMMARY

In accordance with one example, a method is provided for applying a drop of a therapeutic to an eye of a user. Reflected light from the eye of the user is measured at a sensor to provide a time series of intensity values. It is determined from the time series of intensity values if the eye has blinked. The drop of the therapeutic is released in response to a determination that the eye has blinked.

In accordance with another example, a system includes an optical proximity sensor that measures reflected light from the eye of the user at a sensor to provide a time series of intensity values and a blink detector that determines if the eye has blinked from the time series of intensity values. An actuator releases the drop of the therapeutic in response to a determination that the eye has blinked.

In accordance with a further example, a method is provided for applying a drop of a therapeutic to an eye of a user. Reflected light from the eye of the user is measured at a sensor to provide a time series of intensity values. It is determined from the time series of intensity values if the eye has blinked by detecting a rising edge within the time series of intensity values and detecting a falling edge within the time series of intensity values that follows the detected rising edge within a threshold time. The drop of the therapeutic is released in response to a determination that the eye has blinked.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a device for automated application of eye drops to an eye of the user;

FIG. 2 illustrates a state diagram representing the logic of one implementation of the blink detector of FIG. 1;

FIG. 3 illustrates another state diagram representing the logic of one implementation of the blink detector of FIG. 1;

FIG. 4 illustrates another example device for automated application of eye drops to an eye of the user;

FIG. 5 illustrates one method for automated delivery of a drop of a therapeutic to an eye of a user;

FIG. 6 illustrates another method for automated delivery of a drop of a therapeutic to an eye of a user; and

FIG. 7 is a schematic block diagram illustrating an example system of hardware components capable of implementing examples of the systems and methods disclosed herein.

### DETAILED DESCRIPTION

As used herein, a “droplet” is a small drop of fluid, and the terms “drop” and “droplet” are used interchangeably to describe such a drop of fluid.

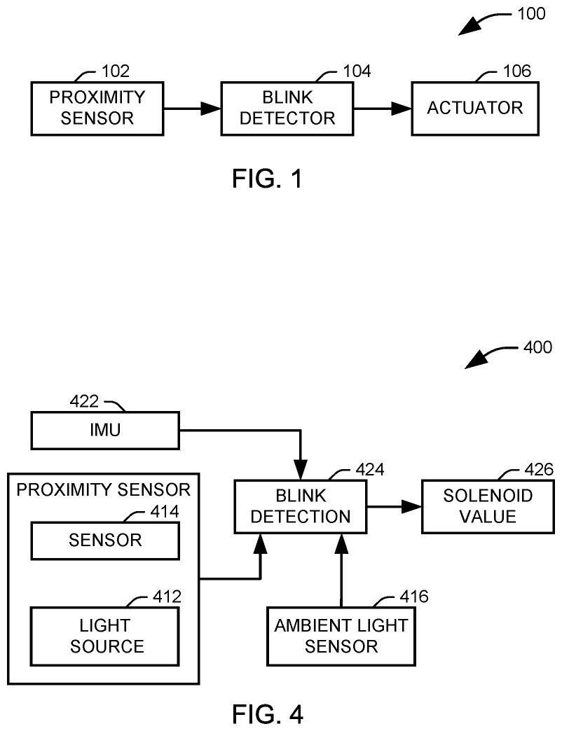

FIG. 1 illustrates a device 100 for automated application of eye drops to an eye of the user. It will be appreciated that the device 100 can be integral with a bottle containing medication intended for application to the user's eye or implemented as a stand-alone device that can be mounted onto a bottle having known dimensions and a known configuration. In one implementation, the device 100 is configured to be attached to a standard prescription eye dropper bottle. The device 100 includes an optical proximity sensor 102 that is positioned to detect reflected light from an eye of the user when the bottle associated with the device is in an appropriate position to deliver mediation to the eye. In the illustrated implementation, the optical proximity sensor 102 includes a light source, such as a light emitting diode, and a photosensor to detect light reflected from the eye, although it will be appreciated that the optical proximity sensor can be configured to operate without active illumination. In one example, the optical proximity sensor 102 is configured, for example, via inclusion of a spectral filter that attenuates light outside of a narrow band of wavelengths, to detect light in the infrared range, for example, in a defined range around a wavelength of either 850 nanometers or 940 nanometers.

A blink detector 104 receives the output of the optical proximity sensor 102 as a series of samples representing the intensity of the detected light, and determines if a blink has occurred. Specifically, the blink detector 104 processes the output of the optical proximity sensor to determine both if the optical proximity sensor 102 is within a threshold distance of the eye (e.g., approximately one inch) from the intensity of the reflected light and to determine when the eye is closing from a change in the intensity of the reflected light. The blink detector 104 can be implemented as software or firmware stored on a non-transitory medium and executed by an associated processor, as dedicated hardware, such as an application specific integrated circuit or field programmable gate array, or as a combination of software and dedicated hardware. It will be appreciated that the wavelength of the light detected at the optical proximity sensor 102 can be selected such that it is invisible to a person so as not to cause distractions or eye strain and also a wavelength at which changes in visible skin color cause minimal changes in reflection. Such a minimum occurs, for example, at 940 nm.

If the light source and photosensor are placed in the correct geometric configuration, most of the reflected light of the cornea from an open eye will have specular reflection away from the aperture of the proximity detector whereas when the eyelid skin is closed over the eye, a strong amount of diffuse reflection off the skin will be coupled into the aperture of the photosensor regardless of the eyelid skin color. Thus a closed eye can have significantly higher captured reflected intensity than the open eye itself, and thus the captured intensity of the reflected light at the optical proximity sensor 102 is increased as the eye closes. In one implementation, the blink detector is configured to detect the rising edge of a peak in reflected light as the eye closes during a blink. It will be appreciated that a blink occurs over a very short period of time, and thus the blink detector 104 can be configured to sample the optical proximity sensor 102 at a very high rate, for example, between one hundred twenty and two hundred hertz, or in some implementations, higher than two hundred hertz.

When a person blinks, after their eyes open, they will not reflexively blink again for the next one-hundred milliseconds. To exploit this window, an actuator 106 can be positioned relative to the bottle to trigger a release of an eye drop when a blink is detected at the blink detector, or more specifically, when the opening of the eye immediately after the blink is detected. Accordingly, the drop can be delivered while the user's natural instinct to blink is suppressed. In one implementation, the actuator 106 can be implemented using a solenoid valve positioned to release a drop in response to a signal provided by the blink detector 104. The actuator 106 can be configured to trigger in approximately ten milliseconds, and the transit time of a drop to the eye at the threshold distance required by the blink detector 104 is approximately ten milliseconds, so in the illustrated example, the blink detector 104 is configured to detect a blink within eighty milliseconds to exploit the one-hundred millisecond window in which instinctual blinking is suppressed. It will be appreciated that the threshold distance can be adjusted to allow for a travel time of the drop that is less than ten milliseconds.

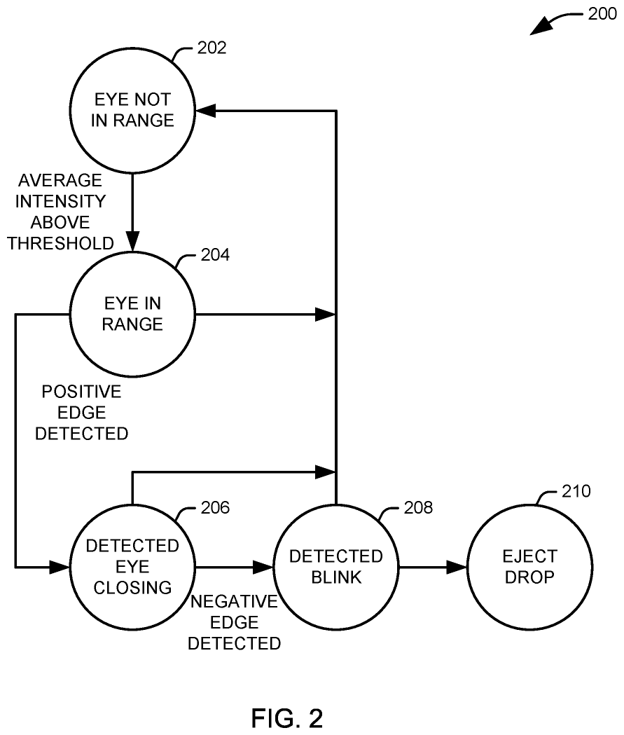

FIG. 2 illustrates a state diagram 200 representing the logic of one implementation of the blink detector 104 of FIG. 1. In the illustrated example, the blink detector detects a blink of the eye and activates the actuator 106 immediately after the eye opens after the blink. In the illustrated implementation, the blink detector maintains a rolling window of samples from the optical proximity sensor 102, that is divided into three contiguous sets of samples, with a most recent set of samples, referred to here as “edge samples,” a second set of oldest samples, referred to here as “steady state samples,” and a third set of samples, between the edge samples and the steady state samples, referred to as “transient samples.” In one example, the rolling window has a length of thirty-three samples, with five edge samples, eleven transient samples, and seventeen steady state samples, although the length of the various sets of samples can vary with the state of the system. As each new sample is received, the rolling window is updated, and a state transition can occur if the appropriate condition is met, although it will be appreciated that a cooldown period can be applied between detection of various events that provoke state transitions, particularly edge detections of a same type (e.g., two positive edges or two negative edges).

The system begins in a first state 202, representing the state in which the eye is not in range. The first state 202 transitions to a second state 204, representing the eye being in range of the eye, when an average value across the values in the rolling window exceeds a threshold value. The second state 204 can transition back to the first state 202 if the average value across the values in the rolling window fails below the threshold value or transition to a third state 206 if a positive edge, that is, a sharp rise in the intensity values, is detected. In one example, a difference between a minimum value of the edge samples and a maximum value of the steady state samples is compared to a threshold value to determine if a positive edge has been detected. The third state 206 represents a first detected closing of the eye. The third state 206 can transition back to the first state 202 if the average value across the values in the rolling window fails below the threshold value or transition to a fourth state 208, representing completion of the blink, if a negative edge, that is, a sharp decline in the intensity values, is detected. In one example, a difference between a maximum value of the steady state samples and a minimum value of the edge samples is compared to a threshold value to determine if a negative edge has been detected. After the detection of the blink at 208, the state transitions to 210, where a drop is released.

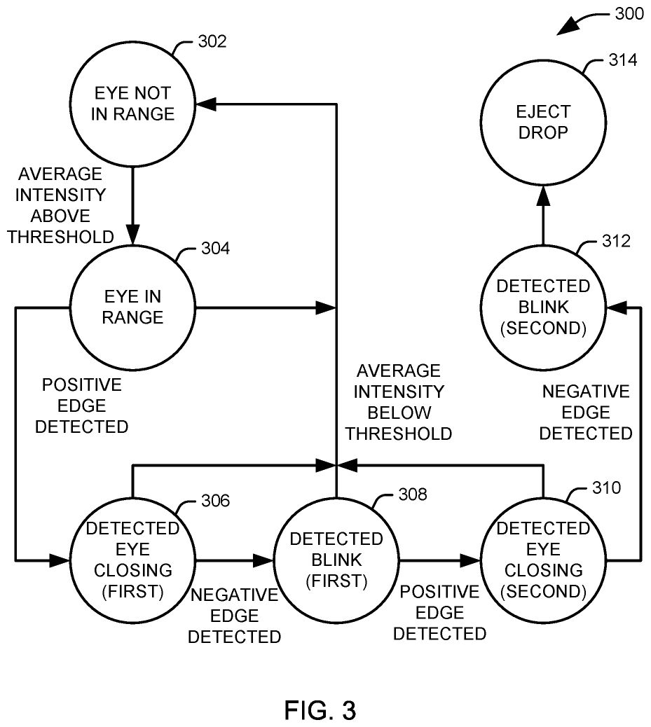

FIG. 3 illustrates a state diagram 300 representing the logic of another implementation of the blink detector 104 of FIG. 1. In the illustrated example, the blink detector detects two blinks of the eye and activates the actuator 106 immediately after the second blink. Similarly to the example of FIG. 2, the blink detector maintains a rolling window of samples from the optical proximity sensor 102, that is divided into three contiguous sets of samples, with a most recent set of samples, referred to here as “edge samples,” a second set of oldest samples, referred to here as “steady state samples,” and a third set of samples, between the edge samples and the steady state samples, referred to as “transient samples.” In one example, the rolling window has a length of thirty-three samples, with five edge samples, eleven transient samples, and seventeen steady state samples, although the length of the various sets of samples can vary with the state of the system. As each new sample is received, the rolling window is updated, and a state transition can occur if the appropriate condition is met, although it will be appreciated that a cooldown period can be applied between detection of various events that provoke state transitions, particularly edge detections of a same type (e.g., two positive edges or two negative edges).

The system begins in a first state 302, representing the state in which the eye is not in range. The first state 302 transitions to a second state 304, representing the eye being in range of the eye, when an average value across the values in the rolling window exceeds a threshold value. The second state 304 can transition back to the first state 302 if the average value across the values in the rolling window fails below the threshold value or transition to a third state 306 if a positive edge, that is, a sharp rise in the intensity values, is detected. In one example, a difference between a minimum value of the edge samples and a maximum value of the steady state samples is compared to a threshold value to determine if a positive edge has been detected. The third state 306 represents a first detected closing of the eye. The third state 306 can transition back to the first state 302 if the average value across the values in the rolling window fails below the threshold value or transition to a fourth state 308 if a negative edge, that is, a sharp decline in the intensity values, is detected. In one example, a difference between a maximum value of the steady state samples and a minimum value of the edge samples is compared to a threshold value to determine if a negative edge has been detected.

The fourth state 308 represents completion of a first blink. The fourth state 308 can transition back to the first state 302 if the average value across the values in the rolling window fails below the threshold value or transition to a fifth state 310 if a positive edge is detected. The fifth state 310 represents a second detected closing of the eye. The third state 306 can transition back to the first state 302 if the average value across the values in the rolling window fails below the threshold value or transition to a sixth state 312, representing a second completed blink, if a negative edge is detected. The actuator is activated immediately at 314 once the system enters the sixth state 312 to deliver the droplet into the eye during the period of suppression of the blink instinct. For the detection of the negative edge in the transition between the fifth state and the sixth state, the number of steady state samples considered can be reduced to twelve, to reduce the delay between detection of the negative edge and the delivery of the drop to the eye.

FIG. 4 illustrates another example device 400 for automated application of eye drops to an eye of the user. It will be appreciated that the device 400 can be integral with a bottle containing medication intended for application to the user's eye or implemented as a stand-alone device that can be mounted onto a bottle having known dimensions and a known configuration, such as a standard prescription eye dropper bottle. The device 400 includes an optical proximity sensor 410 comprising a light source 412 and a sensor 414. It will be appreciated that the light source 412 can be selected to provide light in the infrared range. The light source 412 is positioned to illuminate the eye of the user when the bottle associated with the device is in an appropriate position to deliver mediation to the eye, and the sensor 414 is positioned to detect reflected light from an eye of the user. In the illustrated implementation, the light source 412 provides infrared light with a wavelength of 940 nanometers, and the sensor 414 detects light in a narrow range of wavelengths around 940 nanometers. An inertial measurement unit (IMU) 422 tracks an orientation and acceleration of the device 400 in space relative to a reference direction, for example, the direction of gravitational force.

In addition, an ambient light sensor 416 can detect the amount of natural lighting in a room so as to determine if the measurement is performed inside a room or outside on a clear day. This natural light level typically is reduced when the device is appropriately held near the eye due to some shadowing but can be impacted from the solar background spectrum if outside and in direct sunlight background light in a room or outside. The light source 412 can be implemented as a simple light emitting diode (LED) of a narrow angular range (e.g., +/−30 degrees) so as to be sufficiently directed towards the eye at close distances. However, the light source 412 could also be implemented as an infrared LED or a micro vertical cavity surface emitting laser (VCSEL) that is light safe and also includes time-of-flight functionality. This allows for even more precise distance measurements to the distance to eye but at a higher component cost. An example of this is the Vishay VCNL36826S component.

A blink detector 424 receives the output of the optical proximity sensor 410 as a series of samples representing the intensity of the detected light, as well as the output of the IMU 422 and determines if a blink has occurred. In one example, the blink detector 424 processes the output of the optical proximity sensor 410 to determine both if the optical proximity sensor 410 is within a threshold distance of the eye and if a blink of the eye has occurred. A threshold intensity associated with the appropriate threshold distance may be modified to account for lighting conditions, as determined at the ambient light sensor 316. In some instances, a monolith proximity sensor 410 and ambient sensor 416 are available in a combined single surface mount package, such as those commonly used in cell phones, for example, the Broadcom APDS-9160 surface mount device. In one example, the blink detector 424 detects changes in the detected intensity of the light reflected from the eye to determine a potential blink event and verifies with the data from the IMU 422 that the change in intensity was not caused by movement of the device. Additionally, a potential blink event can be ignored if rapid motion of the device, caused for example, by tremors in the user's hands, is detected at the IMU 422.

In an alternative example, the blink detector 324 comprises a machine learning model that receives the output of the IMU 322 and the optical proximity sensor 310 and outputs the likelihood that the patient has blinked and that a drop is likely to land within the eye if a drop is released at a given time. Accordingly, the output of the machine learning model can represent both the detection of a blink and the stability of the device at the time the blink is detected. In one implementation, the input to the machine learning model can include a time series of values from each of the optical proximity sensor 310 and the IMU 322. In one example, the time series includes the last thirty-three samples from each. In another implementation, the machine learning model receives values derived from recent values outputted from the optical proximity sensor 310 and the IMU 322, including, for example, measures of variation (e.g., variance, standard deviation, range, or interquartile range) and central tendency (e.g., mean or median) for those values. The machine learning model can utilize one or more pattern recognition algorithms, each of which may analyze the data provided by the optical proximity sensor 310 and the IMU 322 to assign a continuous or categorical parameter to the likelihood that a blink has been detected and that a released drop would land in the eye. Where multiple classification or regression models are used, an arbitration element can be utilized to provide a coherent result from the plurality of models. The training process of a given classifier will vary with its implementation, but training generally involves a statistical aggregation of training data into one or more parameters associated with the output class. For rule-based models, such as decision trees, domain knowledge, for example, as provided by one or more human experts, can be used in place of or to supplement training data in selecting rules for classifying a user using the extracted features. Any of a variety of techniques can be utilized for the classification algorithm, including support vector machines (SVM), regression models, self-organized maps, fuzzy logic systems, data fusion processes, boosting and bagging methods, rule-based systems, or artificial neural networks (ANN).

For example, an SVM classifier can utilize a plurality of functions, referred to as hyperplanes, to conceptually divide boundaries in the N-dimensional feature space, where each of the N dimensions represents one associated feature of the feature vector. The boundaries may define a range of feature values associated with each class. Accordingly, a continuous or categorical output value can be determined for a given input feature vector according to its position in feature space relative to the boundaries. In one implementation, the SVM can be implemented via a kernel method using a linear or non-linear kernel. A trained SVM classifier may converge to a solution where the optimal hyperplanes have a maximized margin to the associated features.

An ANN classifier may include a plurality of nodes having a plurality of interconnections. The values from the feature vector may be provided to a plurality of input nodes. The input nodes may each provide these input values to layers of one or more intermediate nodes. A given intermediate node may receive one or more output values from previous nodes. The received values may be weighted according to a series of weights established during the training of the classifier. An intermediate node may translate its received values into a single output according to a transfer function at the node. For example, the intermediate node can sum the received values and subject the sum to a rectifier function. The output of the ANN can be a continuous or categorical output value. In one example, a final layer of nodes provides the confidence values for the output classes of the ANN, with each node having an associated value representing a confidence for one of the associated output classes of the classifier. The confidence values can be based on a loss function such as a cross-entropy loss function. The loss function can be used to optimize the ANN. In an example, the ANN can be optimized to minimize the loss function.

Many ANN classifiers are fully connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A rule-based classifier may apply a set of logical rules to the extracted features to select an output class. The rules may be applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees may be trained on random samples of the training set, and an average (e.g., mean, median, or mode) result across the plurality of decision trees is returned. For a classification task, the result from each tree would be categorical, and thus a modal outcome can be used.

The output of the blink detector 324 is provided to a solenoid valve 326 that releases a drop in response to a determination that a blink has occurred and that a released drop will land in the eye exceeds a threshold value. To ensure that the drop reaches the eye during the one-hundred millisecond period after a blink, the solenoid is configured to be triggered in approximately ten milliseconds. To this end, even before a blink detection event, the solenoid can be electronically prepared to fire without delay by charging up a capacitor such that it is discharged through the solenoid to activate it without delay. A signal provided by the blink detector 324 can be used to quickly turn on a solenoid actuator 326 by electronically turning on a high-speed, high-current transistor switch when the determined threshold value for ejection is reached. This transistor quickly discharges current through the solenoid in a matter of a few milliseconds. Once actuated, the solenoid can mechanically squeeze the bottle releasing a drop of fluid within a few milliseconds far faster than a typical second follow on blink reflex time of eighty milliseconds. This precharging of the discharge capacitor before the blink event is helpful for fast solenoid mechanical actuation with low latency. Allowing for a ten-millisecond transit time for the drop and the ten-millisecond activation of the solenoid, the blink detector 324 is configured to make a determination that a blink has occurred within eighty milliseconds of the eye blinking. By releasing a drop only when a complete blink is detected, including reopening of the eyelids, and while the device is stable in the user's hand, incomplete or wasted applications of the therapeutic can be avoided. This can be particularly helpful for patients with muscle weakness or tremors that might complicate squeezing the bottle while maintaining a suitable alignment of the bottle with the eye.

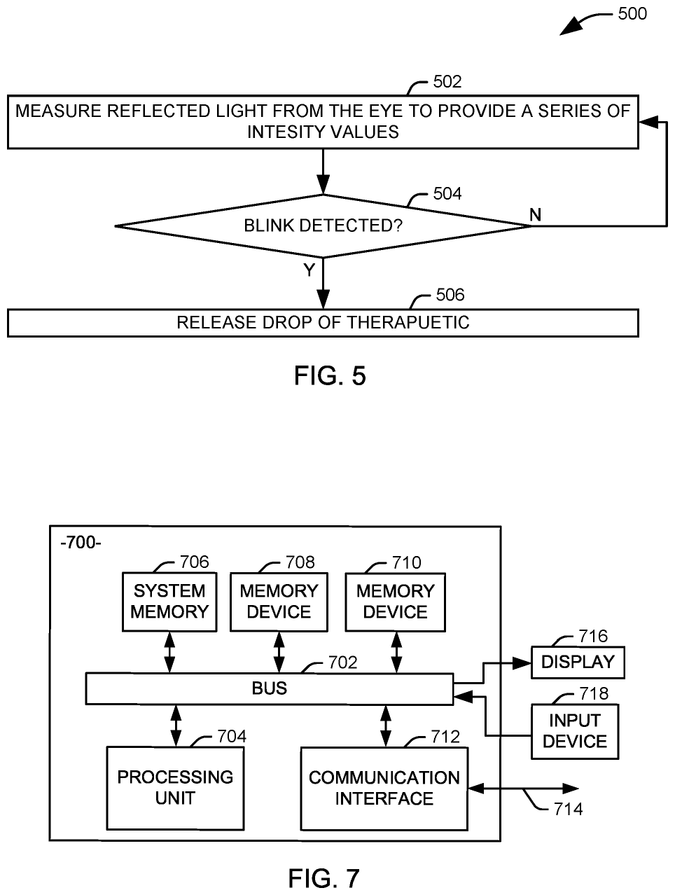

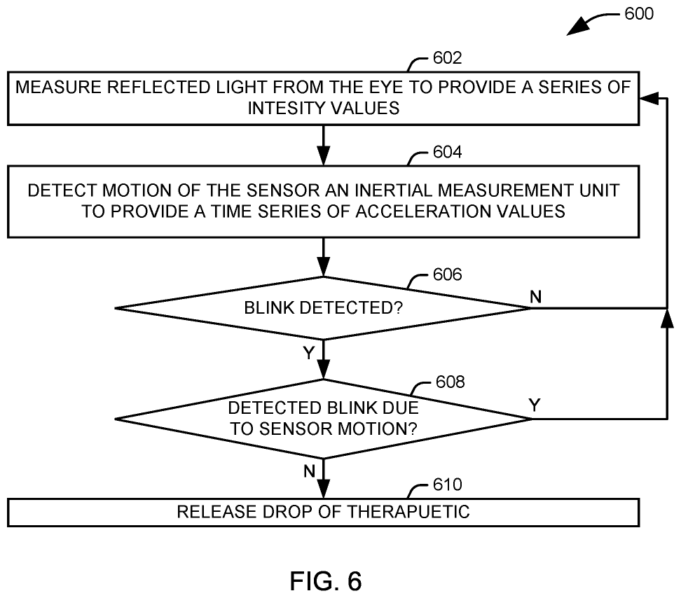

In view of the foregoing structural and functional features described above, example methods will be better appreciated with reference to FIGS. 5 and 6. While, for purposes of simplicity of explanation, the example methods of FIGS. 5 and 6 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method.

FIG. 5 illustrates one method 500 for automated delivery of a drop of a therapeutic to an eye of a user. The method begins at 502, where reflected light from the eye of the user is measured at a sensor to provide a time series of intensity values. In one example, the eye of the user is illuminated with light of a specific wavelength, for example, 940 nanometers, and reflected light within a band of wavelengths including the specific wavelength is detected. At 504, it is determined from the time series of intensity values if the eye has blinked. In some implementations, this detection involves detecting a pattern of blinks, for example, two consecutive blinks. In one example, the time series of intensity values is provided to a machine learning model to determine if a blink has occurred. In another example, a blink is considered to have occurred when a falling edge is detected after a rising edge is detected within the time series of intensity values. If no blink is detected (N), the method 500 returns to 502 to continue monitoring reflected light from the eye. If a blink is detected (Y), a drop of the therapeutic is released at 506. In one example, this is done by activating a solenoid valve in response to the determination that the eye has blinked.

FIG. 6 illustrates another method 600 for automated delivery of a drop of a therapeutic to an eye of a user. The method begins at 602, where reflected light from the eye of the user is measured at a sensor to provide a time series of intensity values. In one example, the eye of the user is illuminated with light of a specific wavelength, for example, 940 nanometers, and reflected light within a band of wavelengths including the specific wavelength is detected. At 604, motion of the sensor is detected at an inertial measurement unit to provide a time series of acceleration values that can be used to rule out hand tremor false signals. At 606, it is determined from the time series of intensity values if the eye has blinked. In some implementations, this detection involves detecting a pattern of blinks, for example, two consecutive blinks. In one example, the time series of intensity values is provided to a machine learning model to determine if a blink has occurred. In another example, a blink is considered to have occurred when a falling edge is detected after a rising edge is detected within the time series of intensity values. If no blink is detected (N), the method returns to 602 to continue monitoring reflected light from the eye. If a blink is detected (Y), it is determined at 608 if the detected eye blink was caused by motion of the sensor from the time series of acceleration values. For example, rapid motion of the sensor by the user can cause a peak in intensity values that resembles a blink simply by moving the target of the sensor from the eye to the surrounding skin. If the blink detection was caused by motion (Y), the method returns to 602 to continue monitoring reflected light from the eye. If the detected blink was not caused by motion (N), a drop of the therapeutic is released at 610. In one example, this is done by activating a solenoid valve in response to the determination that the eye has blinked.

The blink detection algorithms described herein perform very well for the majority of the population, but refinement of the algorithms may be desirable for some people with unique eye shapes or eyelashes. For example, some people with extremely long eyelashes may exhibit unique signatures and classifier signals, especially when it comes to the reflective blink transient signal. The algorithms and classifiers discussed herein can always be tuned to the anatomical aspects of various users using a training algorithm mode incorporated into the firmware of an electronic device, for example, the blink detector associated with the system. The training algorithm mode state of operation does not eject drops for each blink, but instead prompts the user to blink from a different external stimulus such as a buzzer or visible light emitting diode. The device collects repeated blink data for better unique classifier and algorithm training. This can be used, for example, to generate training data for a machine learning model or to determine user-specific parameters for the described edge detection, for example, an threshold interval between detected rising and falling edges or a time between values compared to detect the edge. Accordingly, the algorithm can be trained to a user's unique optical blink transient detection signal.

Further, for the systems and methods discussed above, it is desirable for the user to direct the device to be aligned to their eye, such that the optical proximity sensor is properly directed towards the center of their eye and aligned transversely or in the x-y directions if the z-direction represents the axis aligned with the eye drop ejection. This alignment may be difficult for people who are far-sighted having presbyopia, or difficulty in accommodating to nearby focal distances. Alignment aids, either passive or active, can be built into the sytem to make it much easier for users to obtain adequate alignment to their eye. Such alignment aids could include, for example, magnifying mirrors or alignment LEDs with wavelengths in the visible range that do not interfere with the wavelengths associated with the optical proximity sensor. For example, a green LED could indicate a sufficiently aligned condition when the optical proximity sensor has reached an appropriate threshold indicating alignment the target distance in z and target x-y position has been reached.

FIG. 7 is a schematic block diagram illustrating an example system 700 of hardware components capable of implementing examples of the systems and methods disclosed herein. For example, the system 700 can be used to implement the blink detector of FIG. 1 or 4. The system 700 can include various systems and subsystems. The system 700 can include one or more of a personal computer, a laptop computer, a mobile computing device, a workstation, a computer system, an appliance, an application-specific integrated circuit (ASIC), a server, a server BladeCenter, a server farm, etc.

The system 700 can include a system bus 702, a processing unit 704, a system memory 706, memory devices 708 and 710, a communication interface 712 (e.g., a network interface), a communication link 714, a display 716 (e.g., a video screen), and an input device 718 (e.g., a keyboard, touch screen, and/or a mouse). The system bus 702 can be in communication with the processing unit 704 and the system memory 706. The additional memory devices 708 and 710, such as a hard disk drive, server, standalone database, or other non-volatile memory, can also be in communication with the system bus 702. The system bus 702 interconnects the processing unit 704, the memory devices 706 and 710, the communication interface 712, the display 716, and the input device 718. In some examples, the system bus 702 also interconnects an additional port (not shown), such as a universal serial bus (USB) port.

The processing unit 704 can be a computing device and can include an application-specific integrated circuit (ASIC). The processing unit 704 executes a set of instructions to implement the operations of examples disclosed herein. The processing unit can include a processing core.

The additional memory devices 706, 708, and 710 can store data, programs, instructions, database queries in text or compiled form, and any other information that may be needed to operate a computer. The memories 706, 708 and 710 can be implemented as computer-readable media (integrated or removable), such as a memory card, disk drive, compact disk (CD), or server accessible over a network. In certain examples, the memories 706, 708 and 710 can comprise text, images, video, and/or audio, portions of which can be available in formats comprehensible to human beings.

Additionally, or alternatively, the system 700 can access an external data source or query source through the communication interface 712, which can communicate with the system bus 702 and the communication link 714.

In operation, the system 700 can be used to implement one or more parts of a system in accordance with the present invention. Computer executable logic for implementing the diagnostic system resides on one or more of the system memory 706, and the memory devices 708 and 710 in accordance with certain examples. The processing unit 704 executes one or more computer executable instructions originating from the system memory 706 and the memory devices 708 and 710. The term “computer readable medium” as used herein refers to a medium that participates in providing instructions to the processing unit 704 for execution. This medium may be distributed across multiple discrete assemblies all operatively connected to a common processor or set of related processors.

Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes but is not limited to portable or fixed storage devices, optical storage devices, wireless channels, and/or various other storage mediums capable of storing that contain or carry instruction(s) and/or data.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

In view of the foregoing, the following is claimed:

1. A method for applying a drop of a therapeutic to an eye of a user comprising:
measuring reflected light from the eye of the user at a sensor to provide a time series of intensity values;
determining from the time series of intensity values if the eye has blinked; and
releasing the drop of the therapeutic in response to a determination that the eye has blinked.

2. The method of claim 1, wherein releasing the drop of therapeutic comprises activating a solenoid valve in response to the determination that the eye has blinked.

3. The method of claim 2, in which a current to activate the solenoid valve is prepared for actuation ahead of time by precharging a capacitor before the determination that the eye has blinked such that actuation of the solenoid value is provided by a high speed discharge current with minimal actuation delays.

4. The method of claim 1, wherein releasing the drop of the therapeutic in response to a determination that the eye has blinked, comprises releasing the drop of therapeutic in response to a second determination that the eye has blinked.

5. The method of claim 1, wherein measuring reflected light from the eye of the user comprises illuminating the eye of the user with light of a specific wavelength, and detecting the reflected light within a band of wavelengths including the specific wavelength.

6. The method of claim 1, further comprising:
detecting motion of the sensor at an inertial measurement unit to provide a time series of acceleration values; and
determining if a determination that the eye has blinked was caused by motion of the sensor from the time series of acceleration values;
wherein releasing the drop of the therapeutic in response to the determination that the eye has blinked comprises releasing the drop of the therapeutic in response to the determination that the eye has blinked only if it is determined that the determination that the eye has blinked was not caused by motion of the sensor from the time series of acceleration values.

7. The method of claim 1, wherein determining from the time series of intensity values if the eye has blinked comprises providing the time series of intensity values to a machine learning model.

8. The method of claim 1, wherein determining from the time series of intensity values if the eye has blinked comprises:
detecting a rising edge within the time series of intensity values; and
detecting a falling edge within the time series of intensity values that follows the detected rising edge.

9. The method of claim 1, wherein releasing the drop of the therapeutic in response to the determination that the eye has blinked comprises releasing the drop of therapeutic in response to the determination that the eye has blinked and a determination that the sensor is within a threshold distance of the eye.

10. A system comprising:
an optical proximity sensor that measures reflected light from the eye of the user at a sensor to provide a time series of intensity values;
a blink detector that determines if the eye has blinked from the time series of intensity values; and
an actuator that releases the drop of the therapeutic in response to a determination that the eye has blinked.

11. The system of claim 10, wherein the optical proximity sensor comprises an infrared time-of-flight vertical-cavity surface-emitting laser emitter and a photosensor.

12. The system of claim 10, wherein the optical proximity sensor comprises an infrared light emitting diode and a photosensor.

13. The system of claim 10, wherein the blink detector further determines if the optical proximity sensor is within a threshold distance of the eye by determining if the reflected light has an intensity above a threshold intensity.

14. The system of claim 13, further comprising an ambient light sensor that measures a level of ambient light, the blink detector adjusting the threshold intensity according to the measured level of ambient light.

15. The system of claim 10, wherein the optical proximity sensor measures the reflected light at a rate of at least one hundred twenty hertz.

16. The system of claim 10, further comprising:
a stimulus generator that generates an external stimulus to prompt the user to blink, the optical proximity sensor measuring reflected light from the eye of the user during presentation of the external stimulus to provide an invoked time series of intensity values associated with the blink; and
generating at least one parameter for the blink detector from the invoked time series of intensity values.

17. The system of claim 16, wherein the blink detector comprises a machine learning model that determines if the eye has blinked from the time series of intensity values, and the at least one parameter represents a training sample comprising the invoked time series of intensity values.

18. The system of claim 10, further comprising an inertial measurement unit that detects motion of the optical proximity sensor to provide a time series of acceleration values, the blink detector determining if the determination that the eye has blinked was caused by motion of the sensor from the time series of acceleration values.

19. The system of claim 10, further comprising an alignment aid that assists the user in aligning the optical proximity sensor with the eye, the alignment aid comprising one of a magnifying mirror and a light that is positioned to be visible to the user and is responsive to a determination that the optical proximity sensor is aligned with the eye.

20. A method for applying a drop of a therapeutic to an eye of a user comprising:
measuring reflected light from the eye of the user at a sensor to provide a time series of intensity values;
determining from the time series of intensity values if the eye has blinked by detecting a rising edge within the time series of intensity values detecting a falling edge within the time series of intensity values that follows the detected rising edge within a threshold time; and
releasing the drop of the therapeutic in response to a determination that the eye has blinked.
